(19) United States
(12) Patent Application Publication
(10) Pub. No.: US 2022/0051758 A1
(43) Pub. Date: Feb. 17, 2022

(54) METHOD AND SYSTEM FOR PREDICTING CONTENTS OF CERIUM, PRASEODYMIUM AND NEODYMIUM COMPONENTS BASED ON VIRTUAL SAMPLES

(71) Applicant: East China Jiaotong University, Nanchang City (CN)

(72) Inventors: Rongxiu LU, Nanchang City (CN); Lulu LAI, Nanchang City (US); Hui YANG, Nanchang City (CN); Jianyong ZHU, Nanchang City (CN); Gang YANG, Nanchang City (CN)

(73) Assignee: East China Jiaotong University, Nanchang City (CN)

(21) Appl. No.: 17/132,251

(22) Filed: Dec. 23, 2020

(30) Foreign Application Priority Data
Aug. 11, 2020 (CN) 202010798131.4

(51) Int. Cl. G16C 20/30; G06F 17/18; G16C 60/00

(52) U.S. Cl. CPC G16C 20/30; G16C 60/00; G06F 17/18

(57) ABSTRACT

The disclosure relates to a method for predicting contents of cerium, praseodymium and neodymium components based on virtual samples and a system thereof. The method comprises: obtaining mixed solution of cerium, praseodymium and neodymium in a rare earth extraction process; extracting an H, an S, and an I color feature of a preprocessed image in an HSI color space to obtain an original data sample; constructing a stochastic configuration network model of the content of neodymium component; performing linear midpoint interpolation on the stochastic configuration network model to obtain virtual data samples; fusing original data samples and virtual data samples; reconstructing stochastic configuration network model by using fused data samples; determining content of neodymium component according to reconstructed stochastic configuration network model; and determining contents of cerium and praseodymium according to the content of neodymium component. The disclosure improves accuracy of multi-component prediction in the rare earth extraction process.

### CLAIM TO FOREIGN PRIORITY

The present application claims the priority of Chinese Patent Application No. 202010798131.4, entitled “Method and System for Predicting Contents of Cerium, Praseodymium and Neodymium Components Based on Virtual Samples” filed with the Chinese Patent Office on Aug. 11, 2020, which is incorporated herein by reference in its entirety.

### TECHNICAL FIELD

The disclosure relates to the field of multi-component prediction in a rare earth extraction process, in particular to a method and system for predicting contents of cerium, praseodymium and neodymium components based on virtual samples.

### BACKGROUND

Rare earth comprises 17 elements such as lanthanides, scandium and yttrium, and exist in a form of paragenetic mineral. A cascade extraction separation process is mainly used for the purification of rare earth elements. In the rare earth cascade extraction process, the rare earth elements included in solution to be separated include Ce, Pr and Nd. According to a setting requirement of a production line and complexation degree among the element, extraction agent and detergent, Nd is an easily-extracted component. That is, purple-red extraction liquid rich in Nd ions appears at an outlet of a washing section. Correspondingly, Ce and Pr are difficultly extracted components, that is, apple green raffinate rich in Ce and Pr ions appears at an outlet of an extraction section. A rapid detection of component contents can be achieved by using a correlation between the component contents and color features of Pr and Nd ions in a CePr/Nd extraction production process to establish a component content soft-sensing model. However, at the rare earth extraction production site, small samples due to big difficulty and high cost of data collection cause an inaccurate component content measurement according to the color of the rare earth solution by using a component content prediction model.

### SUMMARY

The purpose of the disclosure is to provide a method and a system for predicting contents of cerium, praseodymium and neodymium components based on virtual samples, to improve the accuracy of multi-component prediction in the rare earth extraction process.

In order to achieve the above purpose, the disclosure provides the following solutions:

A method for predicting contents of cerium, praseodymium and neodymium components based on virtual samples, comprises:

obtaining mixed solution of cerium, praseodymium and neodymium in a rare earth extraction process;

determining an image of the mixed solution according to the mixed solution;

preprocessing the image; wherein the preprocessing comprises background segmentation and filtering;

extracting an H color feature, an S color feature, and an I color feature of the preprocessed image in an HSI color space to obtain an original data sample; wherein the original data sample comprises the H color feature, the S color feature, the I color feature, and a content of neodymium component;

constructing a stochastic configuration network model of the content of neodymium component by taking the H color feature, the S color feature, and the I color feature of the original data sample as input variables, and taking the content of neodymium component of the original data sample as an output variable;

performing linear midpoint interpolation on the stochastic configuration network model to obtain virtual data samples;

fusing the original data samples and the virtual data samples;

reconstructing the stochastic configuration network model by using fused data samples;

determining the content of neodymium component according to the reconstructed stochastic configuration network model; and

determining the contents of cerium and praseodymium according to the content of the neodymium component.

Optionally, constructing the stochastic configuration network model of the content of neodymium component by taking the H color feature, the S color feature, and the I color feature of the original data sample as the input variables, and taking the content of neodymium component of the original data sample as the output variable further comprises:

determining a network output of the stochastic configuration network model by using Y=H_{L}·β; wherein, Y is the network output of the stochastic configuration network model, H_{L} is a hidden layer output matrix corresponding to an L^{th} hidden layer node, and β is a connection weight between a hidden layer and an output layer.

Optionally, performing the linear midpoint interpolation on the stochastic configuration network model to obtain the virtual data samples further comprises:

determining a correspondence between the hidden layer and the network output of the stochastic configuration network model;

performing the linear midpoint interpolation on a hidden layer output and the network output according to the correspondence, to obtain a hidden layer output matrix after the linear midpoint interpolation and a network output matrix after the linear midpoint interpolation; wherein the network output after the linear midpoint interpolation is taken as virtual output data;

determining virtual input data by using a formula of X′=(wⁱⁿ)^{†}(φ⁻¹(o′ₕ)−b); wherein, (wⁱⁿ)⁵⁵⁴ is a generalized inverse of an input weight matrix, b is a bias of a hidden layer neuron, φ⁻¹(·) is an inverse of an activation function, and o′ₕ is the hidden layer output after the linear midpoint interpolation; and

determining the virtual input data and the virtual output data as the virtual data samples.

Optionally, determining the correspondence between the hidden layer and the network output of the stochastic configuration network model further comprises:

determining an output matrix of the hidden layer by using a formula of

$${o}_{h} = φ ( {w}^{i n} \cdot x + b ) = [ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ,$$

wherein, o^{h} is the output matrix of the hidden layer, oₕᵢⱼ is an element in the i^{th} row and the j^{th} column of matrix oₕ,φ(·) is the activation function, wⁱⁿ is the input weight matrix, and x is the input variables; and

determining the correspondence between the hidden layer output and the network output by using a formula of

$$[ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ⟹ [ \begin{matrix}{Y}_{1} \\ {Y}_{2} \\ ⋮ \\ {Y}_{t}\end{matrix} ] .$$

A system for predicting contents of cerium, praseodymium and neodymium components based on virtual samples, comprises:

a mixed solution obtaining module, configured for obtaining mixed solution of cerium, praseodymium and neodymium in a rare earth extraction process;

a mixed solution image determining module, configured for determining an image of the mixed solution according to the mixed solution;

a preprocessing module, configured for preprocessing the image; wherein the preprocessing comprises background segmentation and filtering;

an original data sample determining module, configured for extracting an H color feature, an S color feature, and an I color feature of the preprocessed image in an HSI color space to obtain an original data sample; wherein the original data sample comprises the H color feature, the S color feature, the I color feature, and a content of neodymium component;

a stochastic configuration network model constructing module, configured for constructing a stochastic configuration network model of the content of neodymium component by taking the H color feature, the S color feature, and the I color feature of the original data sample as input variables, and taking the content of neodymium component of the original data sample as an output variable;

a virtual data sample determining module, configured for performing linear midpoint interpolation on the stochastic configuration network model to obtain virtual data samples;

a data fusion module, configured for fusing the original data samples and the virtual data samples;

a reconstruction module, configured for reconstructing the stochastic configuration network model by using fused data samples;

a neodymium component content determining module, configured for determining the content of neodymium component according to the reconstructed stochastic configuration network model; and

a cerium and praseodymium component content determining module, configured for determining the contents of cerium and praseodymium according to the content of the neodymium component.

Optionally the stochastic configuration network model constructing module further comprises:

a network output determining unit, configured for determining a network output of the stochastic configuration network model by using Y=H_{L}·β; wherein, Y is the network output of the stochastic configuration network model, H_{L} is a hidden layer output matrix corresponding to an L^{th} hidden layer node, and β is a connection weight between a hidden layer and an output layer.

Optionally, the virtual data sample determining module further comprises:

a correspondence determining unit, configured for determining a correspondence between the hidden layer and the network output of the stochastic configuration network model;

a linear midpoint interpolation processing unit, configured for performing the linear midpoint interpolation on a hidden layer output and the network output according to the correspondence, to obtain a hidden layer output matrix after the linear midpoint interpolation and a network output matrix after the linear midpoint interpolation; wherein the network output after the linear midpoint interpolation is taken as virtual output data;

a virtual input data determining unit, configured for determining virtual input data by using a formula of X′=(wⁱⁿ)^{†}(φ⁻¹(o′ₕ)−b); wherein, (wⁱⁿ)^{†} is a generalized inverse of an input weight matrix, b is a bias of a hidden layer neuron, φ⁻¹(·) is an inverse of an activation function, and o′ₕ is the hidden layer output after the linear midpoint interpolation; and

a virtual data sample determining unit, configured for determining the virtual input data and the virtual output data as the virtual data samples.

Optionally the correspondence determining unit further comprises:

a hidden layer output matrix determining subunit, configured for determining an output matrix of the hidden layer by using a formula of

$${o}_{h} = φ ( {w}^{i n} \cdot x + b ) = [ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ,$$

wherein, oₕ is the output matrix of the hidden layer, oₕᵢⱼ is an element in the i^{th} row and the j^{th} column of matrix oₕ,φ(·) is the activation function, wⁱⁿ is the input weight matrix, and x is the input variables; and

a correspondence determining subunit, configured for determining the correspondence between the hidden layer output and the network output by using a formula of

$$[ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ⟹ [ \begin{matrix}{Y}_{1} \\ {Y}_{2} \\ ⋮ \\ {Y}_{t}\end{matrix} ] .$$

According to the specific embodiments provided by the disclosure, the disclosure can achieve the following technical effects:

The disclosure provides a method and a system for predicting contents of cerium, praseodymium and neodymium components based on virtual samples. This method increases the number of data samples by adding virtual data samples obtained by constructing the stochastic configuration network model of the content of neodymium component, thereby solving the small sample problem caused by big difficulty and high cost of data collection at the rare earth extraction production site. Further, this disclosure solves the problem of deviation in detection of multi-component content of the rare earth elements with color feature by fusing the original data sample and the virtual data sample, reconstructing the stochastic configuration network model by using fused data samples, and determining the content of neodymium component according to the reconstructed stochastic configuration network model. The disclosure can more accurately predict the content of CePr/Nd components, and has very important practical significance for realizing the accurate detection process of the component content in the rare earth extraction separation process.

### BRIEF DESCRIPTION OF THE DRAWINGS

In order to explain the embodiments of the disclosure or the technical solutions in the prior art more clearly, the drawings needed in the embodiments will be introduced briefly in the following. Obviously, the drawings in the following description are only some embodiments of the disclosure. Other drawings can be obtained from these drawings for those ordinary skill in the art without creative work.

FIG. 1 illustrates a schematic flow chart of a method for predicting contents of cerium, praseodymium and neodymium components based on virtual samples provided by the disclosure;

FIG. 2 (a) is a graph showing a relationship between a first moment of an H component and content of the Nd component, FIG. 2 (b) is a graph showing a relationship between a first moment of an S component and the content of the Nd component, and FIG. 2 (c) is a graph showing a relationship between a first order of an I component and the content of the Nd component;

FIG. 3 illustrates a process of linear midpoint interpolation of the hidden layer output in the i^{-th} row and the j^{-th} row;

FIG. 4 illustrates a flow chart of virtual data sample generation;

FIG. 5 illustrates a block diagram of a prediction principle of the contents of CePr/Nd components;

FIG. 6 is a diagram of accuracy results of an LSSVM model and a stochastic configuration network model with or without virtual samples;

FIG. 7 is a diagram showing relative error test performance of the LSSVM model and the stochastic configuration network model with or without virtual samples;

FIG. 8 illustrates a schematic structural diagram of a system for predicting contents of cerium, praseodymium and neodymium components based on virtual samples provided by the disclosure.

### DETAILED DESCRIPTION OF THE EMBODIMENTS

The technical solutions in the embodiments of the disclosure will be clearly and completely described below in combination with the accompanying drawings in the embodiments of the disclosure. Obviously, the described embodiments are only a part of the embodiments of the disclosure, rather than all the embodiments. Based on the embodiments of the disclosure, all other embodiments obtained by those ordinary skill in the art without creative work shall fall within the protection scope of the disclosure.

The purpose of the disclosure is to provide a method and a system for predicting contents of cerium, praseodymium and neodymium components based on virtual samples, to improve the accuracy of multi-component prediction in the rare earth extraction process.

In order to make the above-mentioned purposes, features and advantages of the disclosure more obvious and easy to understand, the disclosure will be further described in detail below in combination with the accompanying drawings and specific embodiments.

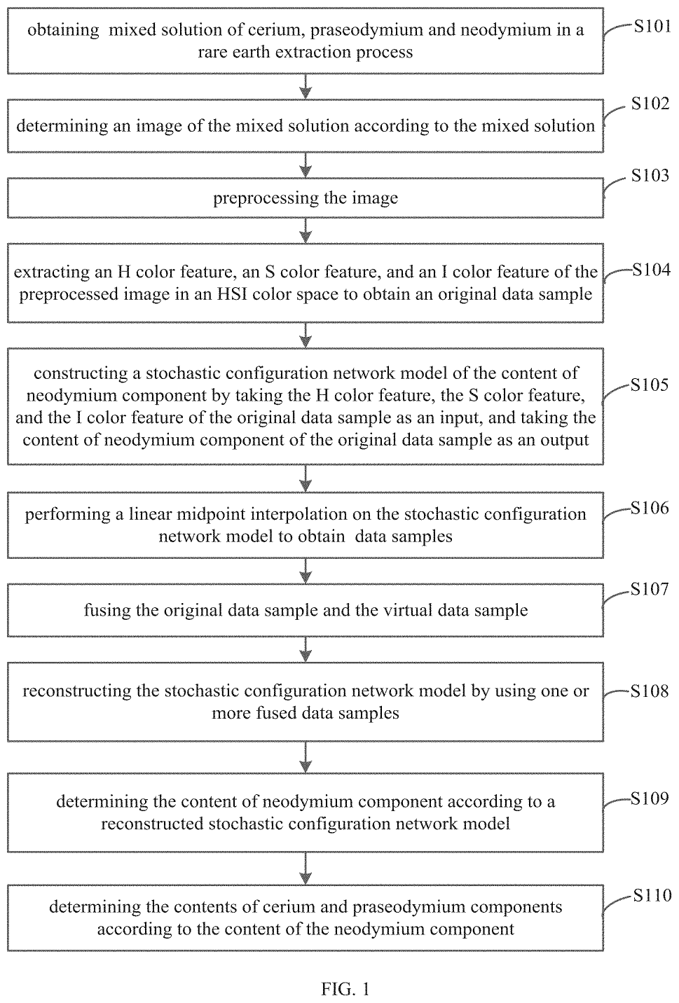

FIG. 1 illustrates a schematic flow chart of the method for predicting contents of cerium, praseodymium and neodymium components based on virtual samples provided by the disclosure. As shown in FIG. 1, the method for predicting contents of cerium, praseodymium and neodymium components based on virtual samples, comprises steps S101-110:

S101: mixed solution of cerium, praseodymium and neodymium in a rare earth extraction process is obtained, wherein, the mixed solution of cerium, praseodymium and neodymium is obtained from a monitoring-level extraction tank in the extraction section and the washing section of the rare earth production site to obtain the component content;

S102: an image of the mixed solution is determined according to the mixed solution, wherein the image is determined by using a rare earth mixed solution video image acquisition system;

S103: the image is pre-processed, wherein the preprocess comprises background segmentation and filtering.

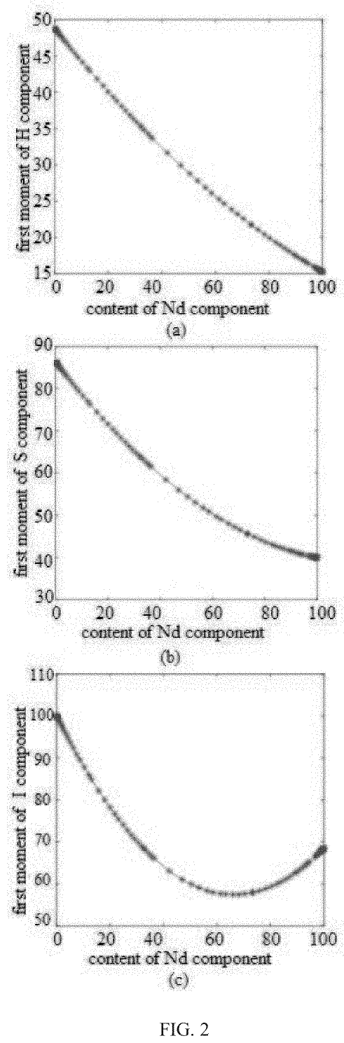

S104: an H color feature, an S color feature, and an I color feature of the preprocessed image in an HSI color space are extracted to obtain an original data sample, wherein the original data sample comprises the H color feature, the S color feature, the I color feature, and the content of neodymium component, and the relationships between the content of neodymium component and the first moment of the H, S, and I components are shown in FIG. 2; and

S105: the H color feature, the S color feature, and the I color feature of the original data sample are taken as input variables, and the content of neodymium component of the original data sample is taken as an output variable, to construct a stochastic configuration network model of the content of neodymium component.

S105 specifically includes:

Determining a network output of the stochastic configuration network model by using Y=H_{L}·β; wherein, Y is the network output of the stochastic configuration network model, H_{L} is a hidden layer output matrix corresponding to the L^{th} hidden layer node, and β is a connection weight between a hidden layer and an output layer.

Wherein, before determining the network output of the stochastic configuration network model, the method further includes:

Giving an objective function of the stochastic configuration network model as ƒ:Y∈R^{M×1}→X={x_{H},x_{S},x_{I}}∈R^{M×3}, assuming that the stochastic configuration network model has L-1 hidden layer nodes, the network output Y_{L-1} can be obtained by the above formula:

Y_{L-1}=Σₗ₌₁^{L-1}βₗφₗ(wₗ^{T}X+bₗ)(L=1,2, . . . , Y₀=0). Wherein, X is an input variable, Y₀ is the objective function, φₗ(·) is an activation function, wₗ is an input weight of the network for the l^{th} hidden layer node, bₗ is a threshold of the network for the l^{th} hidden layer node, and βₗ is an output weight of the network for the l^{th} hidden layer node.

The hidden layer of the network uses the Sigmoid function as the activation function. Then, the output matrix of the hidden layer H_{L} corresponding to the L^{th} hidden layer node is represented as H_{L}=φ(w_{L}^{T}·X)+[b_{L},b_{L}, . . . b_{L}]_{1×N}; where,

$$φ ( x ) = \frac{1}{1 + {e}^{- x}} ,$$

and “·” represents dot product.

Furthermore, the network output of the stochastic configuration network model can be determined by using Y=H_{L}·β. Wherein β can be obtained by the Moore-Penrose generalized inverse solution, that is, β=H_{L}^{†}Y, where H_{L}^{†} represents the pseudo-inverse of the output matrix of the hidden layer H_{L}.

S106: a linear midpoint interpolation is performed on the stochastic configuration network model to obtain virtual data samples.

S106 specifically includes:

Determining a correspondence between the hidden layer and the network output of the stochastic configuration network model; determining the output matrix of the hidden layer by using the formula of

$${o}_{h} = φ ( {w}^{m} \cdot x + b ) [ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ,$$

wherein, oₕ is the output matrix of the hidden layer, oₕᵢⱼ is an element in the i^{th} row and the j^{th} column of the matrix oₕ,φ(·) is the activation function, wⁱⁿ is the input weight matrix, and x is the input variables; and determining the correspondence between the hidden layer output and the network output by using the formula of

$$[ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ⟹ [ \begin{matrix}{Y}_{1} \\ {Y}_{2} \\ ⋮ \\ {Y}_{t}\end{matrix} ] ,$$

performing the linear midpoint interpolation on the hidden layer output and the network output according to the correspondence, to obtain a hidden layer output matrix after the linear midpoint interpolation and a network output matrix after the linear midpoint interpolation; wherein the network output after the linear midpoint interpolation is taken as virtual output data.

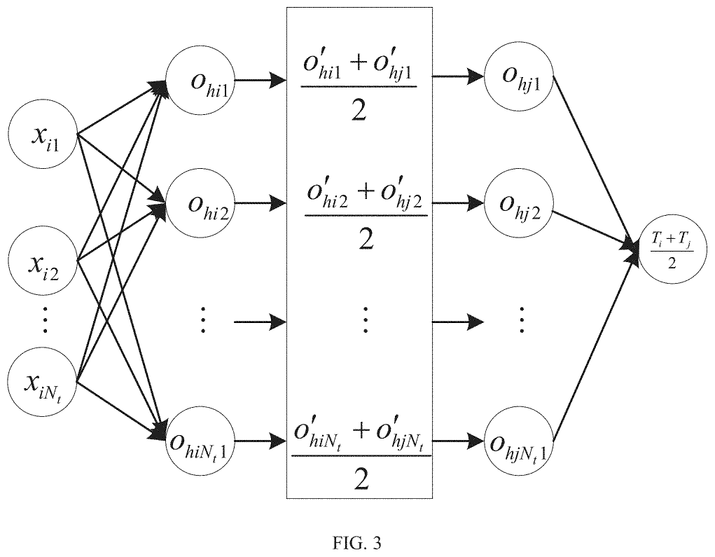

FIG. 3 illustrates the process of linear midpoint interpolation of the hidden layer output in the i^{th} row and the j^{th} row. As shown in FIG. 3, first, a starting row of the interpolation position c_{q}(q=1,2, . . . N) can be determined. And then an euclidean distance d_{q} based on input variable between the starting row and each of rows of the hidden layer matrix can be calculated. An optimal euclidean distance can be selected as an end point of the interpolation c_{q+1}, where the euclidean distance similarity criterion is:

$${d}_{q} = \sqrt{{( {x}_{{H}_{q + 1}} - {x}_{{H}_{q}} )}^{2} + {( {x}_{{S}_{q + 1}} - {x}_{{S}_{q}} )}^{2} + {( {x}_{{I}_{q + 1}} - {x}_{{I}_{q}} )}^{2}} .$$

Assuming that the first row of the output matrix of the hidden layer is the interpolation starting point, and the row with the optimal euclidean distance from the first row is the second row of the output matrix of the hidden layer, that is,

$$[ \begin{matrix}{o}_{h 11} & {o}_{h 12} & … & {o}_{h 1 L} \\ {o}_{h 21} & {o}_{h 22} & … & {o}_{h 2 L}\end{matrix} ] ,$$

the result of the hidden layer output after the linear midpoint interpolation is:

$${o}_{h}^{′} = [ \begin{matrix}{o}_{h 11} & {o}_{h 12} & … & {o}_{h 1 L} \\ \frac{{o}_{h 11} + {o}_{h 21}}{2} & \frac{{o}_{h 12} + {o}_{h 22}}{2} & … & \frac{{o}_{h 1 L} + {o}_{h 2 L}}{2} \\ {o}_{h 21} & {o}_{h 22} & … & {o}_{h 2 L}\end{matrix} ]$$

The network output after the linear midpoint interpolation is

$${Y}^{′} = [ \frac{{Y}_{1} + {Y}_{2}}{2} ] ,$$

where Y′ is the network output after the linear midpoint interpolation , i.e. the virtual output data.

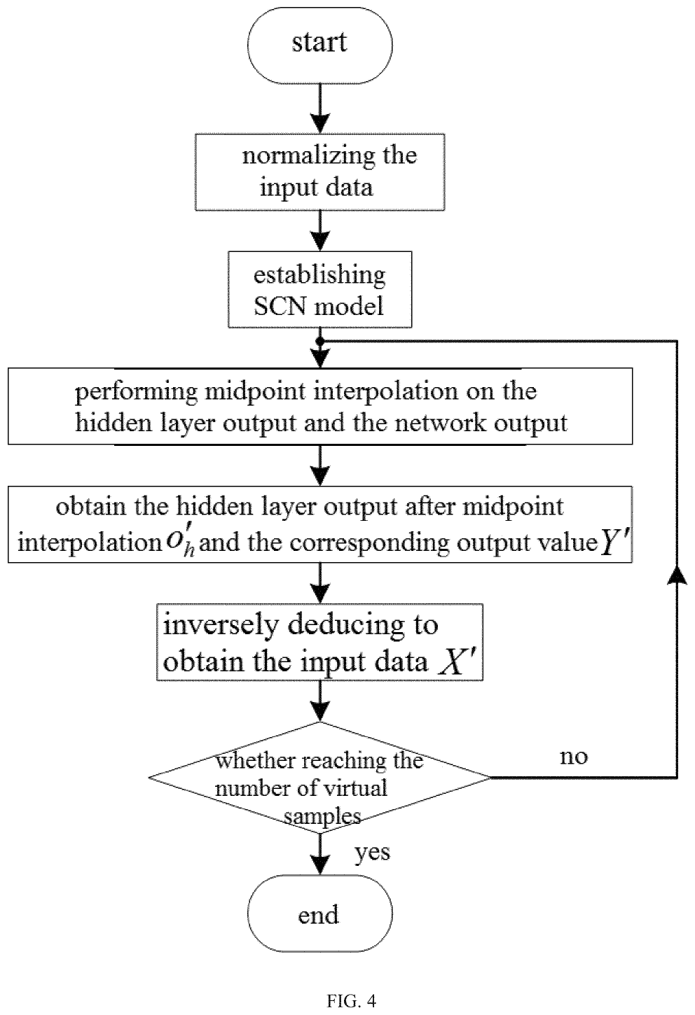

Virtual input data are determined by using the formula of X′=(wⁱⁿ)^{†}(φ⁻¹(o′ₕ)−b), as shown in FIG. 4. Herein, (wⁱⁿ)^{†} is a generalized inverse of the input weight matrix, b is a bias of the hidden layer neurons, φ⁻¹(·) is an inverse of the activation function, o′ₕ is the hidden layer output after the linear midpoint interpolation,

$${φ}^{- 1} ( x ) = ln ( \frac{x}{1 - x} ) , \qquad$$

and (wⁱⁿ)^{†}=((wⁱⁿ)^{T}wⁱⁿ)⁻¹(wⁱⁿ)^{T}.

The virtual input data and the virtual output data are determined as a virtual data sample. The number of samples can be increased by repeatedly using the above steps as needed.

In summary, N virtual samples can be generated after N hidden layer output linear interpolations:

Sᵥ=(X′ᵢ,Y′ᵢ)(i=1,2, . . . , N).

S107: original data samples and the virtual data samples are fused.

S108: the stochastic configuration network model is reconstructed by using fused data samples.

S109: the content of neodymium component is determined according to the reconstructed stochastic configuration network model.

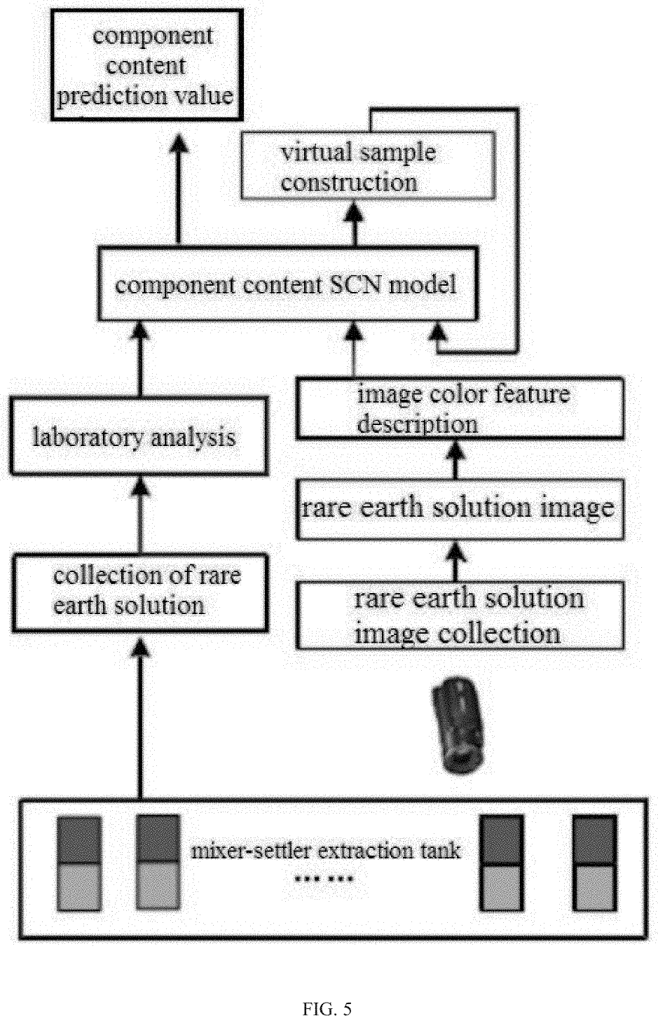

S110: contents of cerium and praseodymium components are determined according to the content of the neodymium component. Wherein, the prediction principle of CePr/Nd component content is shown in FIG. 5.

In order to verify the accuracy of the composition contents of CePr/Nd predicted by the disclosure, the stochastic configuration network model method of the disclosure is compared with the LSSVM method suitable for small sample modeling. A root mean square error and a relative error are taken as two evaluation indicators to evaluate the reconstructed stochastic configuration network model, to verify the validity of the generated virtual sample and the accuracy of the component content prediction model.

As a specific embodiment, relying on the cerium-praseodymium/neodymium extraction industrial production line of the Rare Earth Company, 102 sample solutions were obtained in the monitoring-level extraction tank of the extraction section and the washing section under different working conditions at different times. The mixed solutions were marked and divided into two parts, wherein one part is used to obtain the content of Nd component by using an offline laboratory detection method, and the other part is used to photograph the rare earth solution samples in the laboratory standard light source box to obtain 102 solution images and extract the H, S and I color feature components of the solution images. The first moment of the color feature component and the content of Nd component form the original sample data set. Fifty groups of original samples are stochasticly selected as training samples, and remaining 15 groups are used as test samples. By generating virtual samples, a stochastic configuration network model constructed based on virtual samples is established. In order to illustrate the accuracy of the model and verify the effectiveness of the generated virtual samples, the disclosure sets the following two experiments:

Experiment 1: Taking the first moments of the H, S, and I feature components as the input variables, and the Nd component content as the output variable, 65 groups of original samples are taken to establish the LSSVM model and the stochastic configuration network model of the Nd component content respectively, by using the LSSVM soft-sensing method suitable for small sample modeling and the stochastic configuration network model method. Wherein parameters of the LSSVM model are set as: regularization parameter gam=234.0409, and the parameter value of the kernel function sig2=0.5250826, and parameters of the stochastic configuration network model are set as: the maximum number of hidden layer nodes L=50, and tolerable error ε=0.0001.

Experiment 2: Based on the stochastic configuration network model of Experiment 1, 10 virtual samples was sequentially increased to perform 5 experiments to observe the accuracy change of the stochastic configuration network model with the virtual samples on constructed component content .

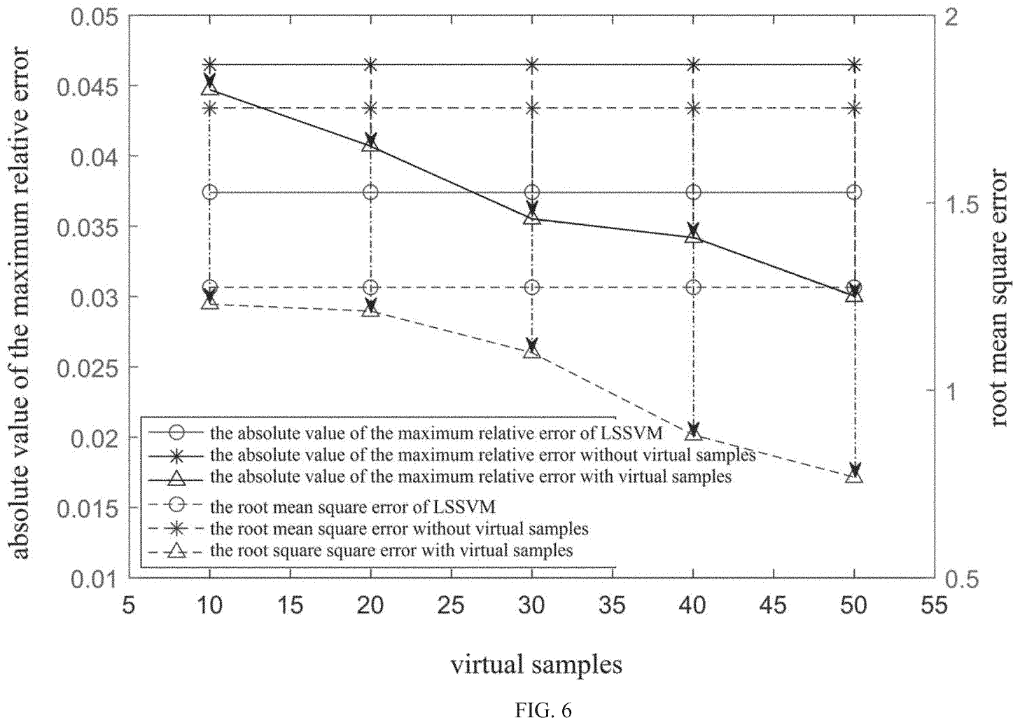

FIG. 6 illustrates the comparison results of the absolute values of the maximum relative errors and the root mean square errors of the Nd component contents predicted by the LSSVM model and the stochastic configuration network model, with or without virtual samples. It can be seen from FIG. 6 that, when there is no virtual sample, the accuracy of the LSSVM model is higher than that of the stochastic configuration network model. After increasing data for the third time, the maximum relative error and the root mean square error of the stochastic configuration network model are both lower than those of the LSSVM. The results show that the component content stochastic configuration network model with virtual samples generated has better performance than the model without virtual sample; and the more virtual samples generated, a higher accuracy and a better performance the prediction model has.

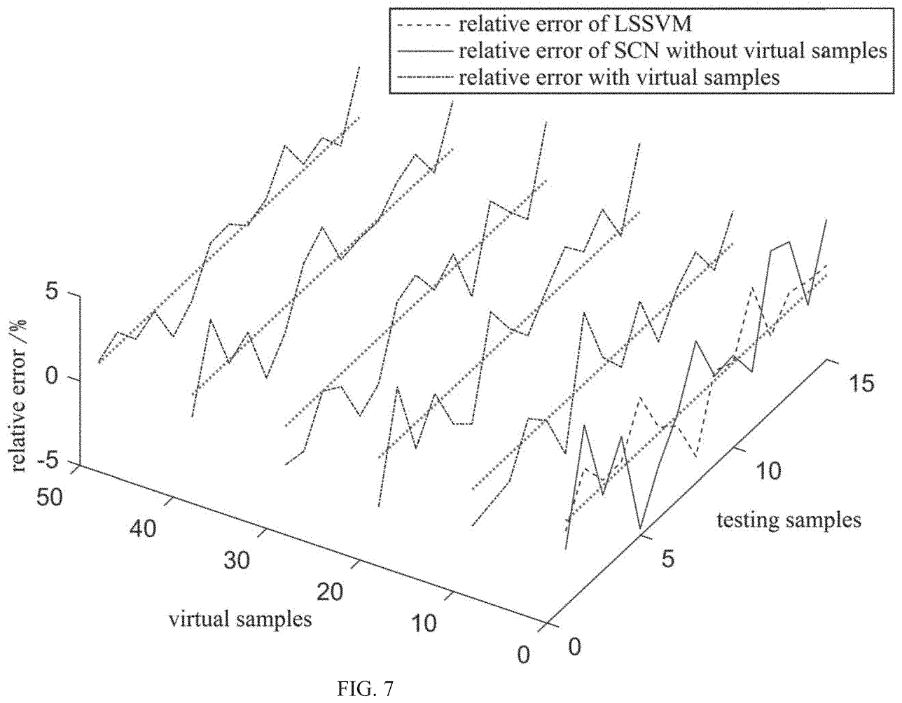

FIG. 7 illustrates the relative error comparison curve of the LSSVM and the stochastic configuration network model with virtual samples and without virtual sample, and increasing different numbers of virtual samples. Wherein, the first curve on the right in FIG. 7 represents the relative errors of the LSSVM and the stochastic configuration network model without virtual samples generated in the test set. The curves from the second curve on the right in FIG. 7 to the left respectively represent the relative errors predicted by the model when the number of virtual samples increases from 10 to 50. It can be seen from FIG. 7 that, compared with a case that the virtual samples are not used, the relative error with virtual samples used more tends to zero, and compared with increasing less virtual samples, the relative error of increasing more virtual samples more tends to zero. That is, as more virtual samples are added, an improvement accuracy of the model is better.

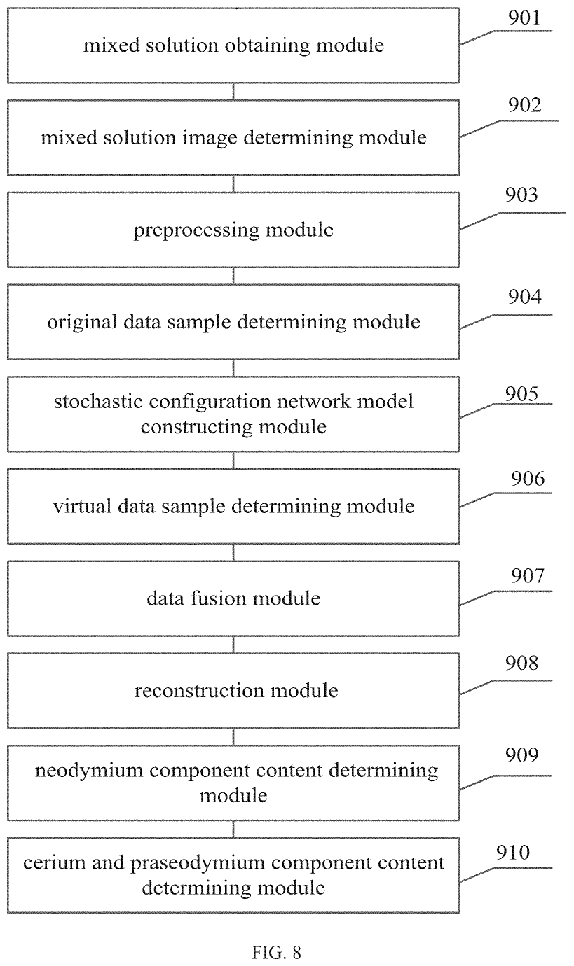

FIG. 8 illustrates a schematic structural diagram of the system for predicting contents of cerium, praseodymium and neodymium components based on virtual samples provided by the disclosure. As shown in FIG. 8, the system for predicting contents of cerium, praseodymium and neodymium components based on virtual samples comprises: a mixed solution obtaining module 901, a mixed solution image determining module 902, a preprocessing module 903, an original data sample determining module 904, a stochastic configuration network model constructing module 905, a virtual data sample determining module 906, a data fusion module 907, a reconstruction module 908, a neodymium component content determining module 909, and a cerium and praseodymium component content determining module 910.

Wherein, the mixed solution obtaining module 901 is configured for obtaining mixed solution of cerium, praseodymium and neodymium in a rare earth extraction process.

The mixed solution image determining module 902 is configured for determining an image of the mixed solution according to the mixed solution.

The preprocessing module 903 is configured for preprocessing the image; wherein the preprocessing comprises background segmentation and filtering.

The original data sample determining module 904 is configured for extracting an H color feature, an S color feature, and an I color feature of the preprocessed image in an HSI color space to obtain an original data sample; wherein the original data sample comprises the H color feature, the S color feature, the I color feature, and a content of neodymium component.

The stochastic configuration network model constructing module 905 is configured for constructing a stochastic configuration network model of the content of neodymium component by taking the H color feature, the S color feature, and the I color feature of the original data sample as input variables, and taking the content of neodymium component of the original data sample as an output variable.

The virtual data sample determining module 906 is configured for performing linear midpoint interpolation on the stochastic configuration network model to obtain virtual data samples.

The data fusion module 907 is configured for fusing the original data samples and the virtual data samples.

The reconstruction module 908 is configured for reconstructing the stochastic configuration network model by using fused data samples.

The neodymium component content determining module 909 is configured for determining the content of neodymium component according to a reconstructed stochastic configuration network model.

The cerium and praseodymium component content determining module 910 is configured for determining the contents of cerium and praseodymium according to the content of the neodymium component.

The stochastic configuration network model constructing module 905 further includes a network output determination unit.

Wherein, the network output determining unit is configured for determining the network output of the stochastic configuration network model by using Y=H_{L}·β; wherein, Y is the network output of the stochastic configuration network model, H_{L} is the hidden layer output matrix corresponding to the L^{th} hidden layer node, and β is the connection weight between a hidden layer and an output layer.

The virtual data sample determining module 906 further includes: a correspondence determining unit, a linear midpoint interpolation processing unit, a virtual input data determining unit, and a virtual data sample determining unit.

Wherein, the correspondence determining unit is configured for determining a correspondence between the hidden layer and the network output of the stochastic configuration network model.

The linear midpoint interpolation processing unit is configured for performing the linear midpoint interpolation on an hidden layer output and the network output according to the correspondence, to obtain a hidden layer output matrix after the linear midpoint interpolation and a network output matrix after the linear midpoint interpolation; wherein the network output after the linear midpoint interpolation is taken as virtual output data.

The virtual input data determining unit is configured for determining virtual input data by using a formula of X′=(wⁱⁿ)^{†}(φ⁻¹(o′ₕ)−b); wherein, (wⁱⁿ)^{†} is a generalized inverse of an input weight matrix, b is a bias of a hidden layer neuron, φ⁻¹(·) is an inverse of an activation function, and o′ₕ is the hidden layer output after the linear midpoint interpolation.

The virtual data sample determining unit is configured for determining the virtual input data and the virtual output data as the virtual data samples.

The correspondence determining unit further includes: a hidden layer output matrix determining subunit and a correspondence determining subunit.

Wherein, the hidden layer output matrix determining subunit is configured for determining an output matrix of the hidden layer by using a formula of

$${o}_{h} = φ ( {w}^{m} \cdot x + b ) [ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ,$$

wherein, oₕ is the output matrix of the hidden layer, oₕᵢⱼ is an element in the i^{th} row and the j^{th} column of matrix oₕ,φ(·) is the activation function, wⁱⁿ is the input weight matrix, and x is the input variables.

The correspondence determining subunit is configured for determining the correspondence between the hidden layer output and the network output by using a formula of

$$[ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ⟹ [ \begin{matrix}{Y}_{1} \\ {Y}_{2} \\ ⋮ \\ {Y}_{t}\end{matrix} ] .$$

The various embodiments in this specification are described in a progressive manner. Each embodiment focuses on the differences from other embodiments, and the same or similar parts between various embodiments can be referred to each other. The system disclosed in the embodiment is described relatively briefly due to the correspondence to the method disclosed in the embodiment, and a relevant description thereof can be referred to the method.

Specific examples are used herein to illustrate the principles and implementations of the disclosure. The description of the above embodiments is only used to help understand the method and core idea of the disclosure. At the same time, those ordinary skill in the art can make some changes in the specific implementation and application scope according to the disclosure. In summary, the content of this specification should not be construed as limiting the disclosure.

What is claimed is:

1. A method for predicting contents of cerium, praseodymium and neodymium components based on virtual samples, comprising:
obtaining mixed solution of cerium, praseodymium and neodymium in a rare earth extraction process;
determining an image of the mixed solution according to the mixed solution;
preprocessing the image; wherein the preprocessing comprises background segmentation and filtering;
extracting an H color feature, an S color feature, and an I color feature of the preprocessed image in an HSI color space to obtain an original data sample; wherein the original data sample comprises the H color feature, the S color feature, the I color feature, and a content of neodymium component;
constructing a stochastic configuration network model of the content of neodymium component by taking the H color feature, the S color feature, and the I color feature of the original data sample as input variables, and taking the content of neodymium component of the original data sample as an output variable;
performing linear midpoint interpolation on the stochastic configuration network model to obtain virtual data samples;
fusing the original data samples and the virtual data samples;
reconstructing the stochastic configuration network model by using fused data samples;
determining the content of neodymium component according to the reconstructed stochastic configuration network model; and
determining the contents of cerium and praseodymium according to the content of the neodymium component.

2. The method for predicting the contents of cerium, praseodymium and neodymium components based on the virtual samples according to claim 1, wherein constructing the stochastic configuration network model of the content of neodymium component by taking the H color feature, the S color feature, and the I color feature of the original data sample as the input variables, and taking the content of neodymium component of the original data sample as the output variable further comprises:
determining a network output of the stochastic configuration network model by using Y=H_{L}·β; wherein, Y is the network output of the stochastic configuration network model, H_{L} is a hidden layer output matrix corresponding to an L^{th} hidden layer node, and β is a connection weight between a hidden layer and an output layer.

3. The method for predicting the contents of cerium, praseodymium and neodymium components based on the virtual samples according to claim 2, wherein performing the linear midpoint interpolation on the stochastic configuration network model to obtain virtual data samples further comprises:
determining a correspondence between the hidden layer and the network output of the stochastic configuration network model;
performing the linear midpoint interpolation on a hidden layer output and the network output according to the correspondence, to obtain a hidden layer output matrix after the linear midpoint interpolation and a network output matrix after the linear midpoint interpolation; wherein the network output after the linear midpoint interpolation is taken as virtual output data;
determining virtual input data by using a formula of X′=(wⁱⁿ)^{†}(φ⁻¹(o′ₕ)−b); wherein, (wⁱⁿ)^{†} is a generalized inverse of an input weight matrix, b is a bias of a hidden layer neuron, φ⁻¹(·) is an inverse of an activation function, and o′ₕ is the hidden layer output after the linear midpoint interpolation; and
determining the virtual input data and the virtual output data as the virtual data samples.

4. The method for predicting the contents of cerium, praseodymium and neodymium components based on the virtual samples according to claim 3, wherein determining the correspondence between the hidden layer and the network output of the stochastic configuration network model further comprises:
determining an output matrix of the hidden layer by using a formula of
$${o}_{h} = φ ( {w}^{m} \cdot x + b ) [ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ,$$
wherein, oₕ is the output matrix of the hidden layer, oₕᵢⱼ is an element in the i^{th} row and the j^{th} column of matrix oₕ,φ(·) is the activation function, wⁱⁿ is the input weight matrix, and x is the input variables; and
determining the correspondence between the hidden layer output and the network output by using a formula of
$$[ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ⟹ [ \begin{matrix}{Y}_{1} \\ {Y}_{2} \\ ⋮ \\ {Y}_{t}\end{matrix} ] .$$

5. A system for predicting the contents of cerium, praseodymium and neodymium components based on the virtual samples, comprising:
a mixed solution obtaining module, configured for obtaining mixed solution of cerium, praseodymium and neodymium in a rare earth extraction process;
a mixed solution image determining module, configured for determining an image of the mixed solution according to the mixed solution;
a preprocessing module, configured for preprocessing the image; wherein the preprocessing comprises background segmentation and filtering;
an original data sample determining module, configured for extracting an H color feature, an S color feature, and an I color feature of the preprocessed image in an HSI color space to obtain an original data sample; wherein the original data sample comprises the H color feature, the S color feature, the I color feature, and a content of neodymium component;
a stochastic configuration network model constructing module, configured for constructing a stochastic configuration network model of the content of neodymium component by taking the H color feature, the S color feature, and the I color feature of the original data sample as input variables, and taking the content of neodymium component of the original data sample as an output variable;
a virtual data sample determining module, configured for performing linear midpoint interpolation on the stochastic configuration network model to obtain virtual data samples;
a data fusion module, configured for fusing the original data samples and the virtual data samples;
a reconstruction module, configured for reconstructing the stochastic configuration network model by using fused data samples;
a neodymium component content determining module, configured for determining the content of neodymium component according to the reconstructed stochastic configuration network model; and
a cerium and praseodymium component content determining module, configured for determining the contents of cerium and praseodymium according to the content of the neodymium component.

6. The system for predicting the contents of cerium, praseodymium and neodymium components based on the virtual samples according to claim 5, wherein the stochastic configuration network model constructing module further comprises:
a network output determining unit, configured for determining a network output of the stochastic configuration network model by using Y=H_{L}·β; wherein, Y is the network output of the stochastic configuration network model, H_{L} is a hidden layer output matrix corresponding to an L^{th} hidden layer node, and β is a connection weight between a hidden layer and an output layer.

7. The system for predicting the contents of cerium, praseodymium and neodymium components based on the virtual samples according to claim 6, wherein the virtual data sample determining module further comprises:
a correspondence determining unit, configured for determining a correspondence between the hidden layer and the network output of the stochastic configuration network model;
a linear midpoint interpolation processing unit, configured for performing the linear midpoint interpolation on a hidden layer output and the network output according to the correspondence, to obtain a hidden layer output matrix after the linear midpoint interpolation and a network output matrix after the linear midpoint interpolation; wherein the network output after the linear midpoint interpolation is taken as virtual output data;
a virtual input data determining unit, configured for determining virtual input data by using a formula of X′=(wⁱⁿ)^{†}(φ⁻¹(o′ₕ)−b); wherein, (wⁱⁿ)^{†} is a generalized inverse of an input weight matrix, b is a bias of a hidden layer neuron, φ⁻¹(·) is an inverse of an activation function, and o′ₕ is the hidden layer output after the linear midpoint interpolation; and
a virtual data sample determining unit, configured for determining the virtual input data and the virtual output data as the virtual data samples.

8. The system for predicting the contents of cerium, praseodymium and neodymium components based on the virtual samples according to claim 7, wherein the correspondence determining unit further comprises:
a hidden layer output matrix determining subunit, configured for determining an output matrix of the hidden layer by using a formula of
$${o}_{h} = φ ( {w}^{m} \cdot x + b ) [ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ,$$
wherein, oₕ is the output matrix of the hidden layer, oₕᵢⱼ is an element in the i^{th} row and the j^{th} column of matrix oₕ,φ(·) is the activation function, wⁱⁿ is the input weight matrix, and x is the input variables; and
a correspondence determining subunit, configured for determining the correspondence between the hidden layer output and the network output by using a formula of
$$[ \begin{matrix}{o}_{h 11} & {o}_{h 12} & … & {o}_{h 1 L} \\ {o}_{h 21} & {o}_{h 22} & … & {o}_{h 2 L} \\ ⋮ & ⋮ & ⋱ & ⋮ \\ {o}_{{hN}_{t} 1} & {o}_{{hN}_{t} 2} & … & {o}_{{hN}_{t} L}\end{matrix} ] ⟹ [ \begin{matrix}{Y}_{1} \\ {Y}_{2} \\ ⋮ \\ {Y}_{t}\end{matrix} ] .$$
